(19) United States
(12) Patent Application Publication
(10) Pub. No.: US 2011/0263438 A1
(43) Pub. Date: Oct. 27, 2011

(54) Diagnosis and complication risk assessment of pancreatic diabetes using procalcitonin

(75) Inventor: Mehmet Ali Soylemez, Istanbul (TR)

(73) Assignee: Mehmet Ali Soylemez, Istanbul (TR)

(21) Appl. No.: 12/931,507

(22) Filed: Feb. 3, 2011

Related U.S. Application Data
(60) Provisional application No. 61/326,728, filed on Apr. 22, 2010.

(51) Int. Cl. C40B 30/00; G01N 33/566; C40B 60/12; C12Q 1/50; C12Q 1/28

(52) U.S. Cl. 506/7; 435/17; 435/28; 506/39; 435/7.92

(57) ABSTRACT

The invention relates to a method for diagnosis and/or diabetic complications and/or risk assessment of pancreatic diabetes, in particular of diabetic sequelae, wherein a determination of the marker procalcitonin (PCT: SEQ ID No. 1) or a partial peptide or fragment thereof or if contained in a marker combination (Panel, Cluster) is carried out on a patient under investigation. The invention further relates to a diagnostic device and a kit for carrying out said method.

The Invention relates to a method for the diagnosis and/or diabetic complications and/or risk stratification of diabetes mellitus, particularly of diabetic sequelae, wherein a determination of the marker procalcitonin (PCT: SEQ ID No. 1) or a partial peptide or fragment thereof, or contained in a marker combination (panel, cluster), is carried out on a patient to be investigated. Furthermore, the invention relates to a diagnostic device and a kit for carrying out the method.

The diagnosis of diabetes mellitus and/or diabetic complications is not known for procalcitonin until our on the bases of procalcitonin genetic expression experimental design and study ((Mehmet Mi Soylemez et al. A Novel Mechanism between Type II Diabetes Mellitus and Procalcitonin Gene Expression Molecular Therapy (2005) 11, S346|[ndash]|S346; doi: 10.1016/j.ymthe.2005.07.437). Relationship between plasma procalcitonin levels and diabetic complications in patients with diabetes mellitus (Mehmet A. Soylemez et al. A Novel Mechanism between Diabetes Mellitus Complications and Procalcitonin Gene Expression. Molecular Therapy (2006) 13, S86|[ndash]|S86; doi: 10.1016/j.ymthe.2006.08.250). However, other diabetic complication follow up markers are disadvantageous, because that the lack of a diabetic complication injury marker of diabetes mellitus, results in no reliable diagnosis can take place. Therefore, there is a need for presenting on the bases of genetic mechanism and a reliable diagnosis of diabetes mellitus, or for undertaking a (risk) stratification, particularly with regard to further clinical decisions and, in particular, with regard to the degree of severity of diabetes mellitus or diabetic sequelae.

Furthermore, the procalcitonin (PCT) determination in diagnosis is described in the state of the art, particularly with regard to an Investigation of sepsis (Yukioka et al., Ann. Acad. Med. Singapore 30: 528-31 (2001). However, the suitability of procalcitonin (PCT: SEQ ID No. 1) for the diagnosis and complications of diabetes mellitus are not disclosed.

It is the task of the present invention to make available an improved method for the diagnosis and/or diabetic complications and/or risk stratification of diabetes mellitus, particularly of diabetic sequelae.

This task is accomplished by means of a method for in vitro diagnosis and/or risk stratification of diabetes mellitus, wherein a determination of the marker procalcitonin (PCT: SEQ ID No. 1) or a partial peptide or fragment thereof, or contained in a marker combination (panel, cluster), is carried out in a patient to be investigated (referred to hereinafter as method according to the invention).

The term “risk stratification,” according to the invention, comprises finding diabetes patients, particularly those having diabetic sequelae, with the worse prognosis, for the purpose of intensive diagnosis and therapy/treatment (of sequelae) of diabetes mellitus, with the goal of allowing as advantageous a course of the diabetes mellitus as possible.

For this reason, it is particularly advantageous that a reliable diagnosis and/or risk stratification can take place by means of the method according to the invention. The method according to the invention allows clinical decisions that lead to a more rapid diagnosis, particularly of the diabetic sequelae and prognosis. Such clinical decisions also comprise further treatment using medications, for the treatment or therapy of diabetes mellitus and complications.

In another preferred embodiment of the method according to the invention, diagnosis and/or risk stratification take place for prognosis, for prophylaxis, for early detection and detection by means of differential diagnosis, for assessment of the degree of severity, and for assessment of the course of diabetes mellitus and complications as an accompaniment to the therapy.

In another preferred embodiment of the method according to the invention, samples of body fluids, particularly blood, optionally whole blood, serum, or available plasma, are taken from the patient to be investigated, and the diagnosis takes place in vitro/ex vivo, i.e. outside of the human or animal body. The diagnosis and/or risk stratification can take place on the basis of the determination of the marker procalcitonin (PCT: SEQ ID No. 1) or partial peptides or fragments thereof, and its amount that is present, or a change in amount, as compared with a reference, in at least one patient sample.

Within the scope of this invention, the term “diabetes mellitus,” particularly Type II diabetes mellitus (insulin-resistant), is understood to mean a chronic inflammatory metabolic disease, where the production of insulin in the beta cells of the islets of Langerhans in the pancreas is disturbed, or insulin is present but cannot correctly act at its target location, the cell membranes. The results of this disturbed insulin production and effect are elevated blood sugar values (hyperglycemia). In the diabetic disease profile, a differentiation is made between prediabetes, in which a “disturbed glucose tolerance,” which can be detected by laboratory chemistry, occurs only in the end stage, and actual manifest diabetes mellitus. Insulin resistance stands at the beginning of the prediabetic illness phase. Almost at the same time, endothelial dysfunction already develops, along with hyperlipoproteinemia and hypertensive dysfunction of the cardiovascular system. The results of this risk constellation are furthermore artherosclerotic changes in the blood vessel walls (microangiopathy and macroanglopathy), as well as vascular complications as the result of microcirculation problems. Other sequelae and concomitant illnesses are diabetic retinopathy going as far as blindness, as well as nephropathy going as far as renal insufficiency, neuropathy, the diabetic foot syndrome, and cardiovascular complications. Diabetic complications is characterized by a chronic process, which commences years before diabetes mellitus becomes overt. Each step of pathogenetic cascade is accompanied by inflammation (Otter W., Standl E., Schnell O, Inflammation and atherogenesis in diabetes mellitus-new therapeutic approaches. Herz. 29(5):524-31, 2004). Procalcitonin is elevated by inflammatory processes, with greater specificity and sensitivity than other acute phase proteins (Hatheril M., Tibby S M., Sykes K., Turner C., Murdoch, I A., Diagnostic markers of infection: comparision of procalcitonin with C reactive protein and leucocyte count. Arch Dis Child). For this reason, the invention relates to the diagnosis and/or risk stratification of Type II diabetes mellitus, and its sequelae and concomitant illnesses, particularly endothelial dysfunction, hyperlipoproteinemia, hypertensive dysregulation of the cardiovascular system, diabetic retinopathy, nephropathy, renal insufficiency, neuropathy, diabetic foot syndrome, and cardiovascular complications.

All the aforementioned indications are furthermore described in Otter W., Standl E., Schnell O., Inflammation and atherogenesis in diabetes mellitus-new therapeutic approaches. Herz. 29(5):524-31, 2004.

Within the scope of this invention, “Procalcitonin (abbreviated as: PCT) was originally described in 1984 as a 116-aminoacid protein with a molecular weight of 14.5 kDa (Le Moullec J. M., et al. The complete sequence of human procalcitonin. FEBS Lett., 167 (1), 93-97 (1984)). The PCT gene, referred to as Calc-1, is located on chromosome 11p15.4 and was sequenced in 1989. The promoter has sites for basal transcription factors but more interestingly, also has sites for NF κβ (Nuclear factor κβ) and AP-1(Activator protein-1), factors induced under inflammatory conditions (Whicher J., Bienvenu J., Monneret G., Procalcitonin as an acute phase marker. Ann Clin Biochem. 38(5):483-93, 2001). Type II diabetes mellitus is associated with oxidative stress and elevation of advanced glycation end products (AGEs). AGEs are produced by a non-enzymatic, Maillard reaction between reducing sugars and either proteins or lipids. AGEs interact the receptor for advanced glycation end products (RAGE) and, RAGE activation is caused by elevation of transcriptional factors NF κβ and AP-1 (Beckman J., Creager M., Libby P., Diabetes and Atherosclerosis JAMA 287: 2570-2581, 2002). These factors (NF κβ and AP-1) induce procalcitonin gene expression (Mehmet Ali Soylemez et al. A Novel Mechanism between Type II Diabetes Mellitus and Procalcitonin Gene Expression Molecular Therapy (2005) 11, S3461[ndash]1S346; doi: 10.1016/j.ymthe.2005.07.437). This algorithmic mechanism was based on genetic expression of procalcitonin and first time showed by our study. Results of our study and experimental design imply procalcitonin as an important mediator during the development of diabetic complications and/or diabetes mellitus. Thus, the results of this study show that procalcitonin (PCT: SEQ ID No. 1) is a new marker for diabetes mellitus and/or diabetic patients with complications (Mehmet Mi Soylemez; ‘Procalcitonin is a specific marker of diabetic complication process’((Control No. 2010-A-377) ESHG conference 2010 Gothenburg, Sweden; EJHG volume 18 supl. 1, Metabolic disorders Page: 352, P 13.14).

Furthermore, the ‘procalcitonin’ according to the invention can demonstrate modifications such as glycolization, lip(o)idiation, or derivatization.

In another embodiment, the determination of procalcitonin (PCT: SEQ ID No. 1) can additionally take place with other markers, where the procalcitonin (PCT: SEQ ID No. 1) is contained in a marker combination (panel, cluster), specifically and preferably with those that already indicate diabetes mellitus. In another particular embodiment, this can also be a vascular marker that can indicate an endothelial dysfunction of the cardiovascular system that accompanies diabetes.

For this reason, the Invention relates to an embodiment of the method according to the invention where the determination is additionally carried out with at least one further marker selected from the group of inflammatory markers, vascular markers, in a patient to be investigated.

According to the invention, the inflammatory marker can be selected from at least one marker of the group of C-reactive protein (CRP), cytokines, such as TNF-alpha, for example, interleukins, such as IL-6, interleukin-1.beta. Proadrenomedullin (proADM), midregional proadrenomedullin (MR-proADM) angiotensin II, endothelin-1, and adhesion molecules, such as VCAM or ICAM, and the vascular marker can be selected from at least one marker of the group of creatine kinase, myeloperoxidase, myoglobin, natriuretic protein, particularly ANP (or ANF), proANP, NT-proANP, BNP, proBNP, NT-proBNP, or a partial sequence thereof, in each instance, CRP. Furthermore, this term is also understood to mean (pro)hormones that regulate the cardiovascular system, particularly such as pro-gastrin-releasing peptide (proGRP), pro-endothelin-1, pro-leptin, pro-neuropeptide-Y, pro-somatostatin, pro-neuropeptide-YY, pro-opiomelanocortin, or a partial sequence thereof, in each instance.

In another embodiment, at least one diabetic marker/factor can additionally be determined. Diabetic markers/factors are, according to the invention, particularly those such as adiponectin, carbohydrates, fats, such as cholesterols (LDH) and others, Body Mass Index (BMI), age, blood pressure, HOMA-IR (Homeostasis Model Assessment-insulin Resistance index, for a determination see: Matthews D R, Hosker J P, Rudenski A S, Naylor B A, Treacher D F, Turner R C, Homeostasis Model Assessment: Insulin Resistance and B-cell Function from Fasting Plasma Glucose and Insulin Concentrations in Man. Diabetologia 28:412-419. 1985).

In another embodiment of the Invention, the method according to the invention can be carried out by means of parallel or simultaneous determinations of the markers (e.g. multi-titer plates with 96 cavities and more); where the determinations are carried out on at least one patient sample.

Furthermore, the method according to the invention and its determinations can be carried out using an automated analysis device, particularly using a Kryptor (http://www.layptor.net/).

In another embodiment, the method according to the Invention and its determinations can be carried out by means of a rapid test (e.g. lateral flow test), whether using single-parameter or multi-parameter determinations.

Furthermore, the Invention relates to the use of procalcitonin (PCT: SEQ ID No. 1) or partial peptides or fragments thereof, or contained in a marker combination (panel, cluster), for in vitro diagnosis and/or diabetic complications and/or risk stratification of diabetes mellitus, particularly Type II diabetes mellitus, and its sequelae and concomitant illnesses, as well as, in particular, taking the aforementioned embodiments into consideration. The marker combination can contain another suitable marker, if necessary.

Another task is making available a corresponding diagnostic device, or the use of such a device for carrying out the methods according to the Invention.

Within the scope of this invention, such a diagnostic device is particularly understood to be an array or assay (e.g. immune assay, ELISA, etc.), in the broadest sense a device for carrying out the method according to the invention.

The invention furthermore relates to a kit or the use of such a kit for in vitro diagnosis or risk stratification of diabetes mellitus, particularly Type II diabetes mellitus, and its sequelae and concomitant illnesses, where a determination of procalcitonin (PCT: SEQ ID No. 1) or partial peptides or fragments thereof, or contained in a marker combination (panel, cluster), is carried out in a patient to be investigated, particularly taking into consideration the aforementioned embodiments. Such detection reagents comprise antibodies, etc. . . .

The following examples and figures serve for a more detailed explanation of the invention, but without restricting the invention to these examples and figures.

### EXAMPLES

### Example 1

Serum procalcitonin levels were analized with kryptor analizator. Kryptor-PCT is a kit designed for kryptor automated immunofluorescent assay of procalcitonin in serum.

Statistical analysis was performed by using SPSS method. The results were expressed as mean±standard deviation. Individuals between group comparisons were made using Student's-T test. Significance was defined at P≦0.05

Procalcitonin was determined in 15 healthy test subjects with undisturbed glucose tolerance, 18 patients having manifest diabetes mellitus Type II without complication (abbreviated as: “DM II”). and 15 diabetics who were suffering from diabetic foot complication

FIG. 2 and Table-2 shows procalcitonin in healthy test subjects, patients having Type II diabetes mellitus (DM II) without complications (FIG. 1, Table-1), and patients having DM II with diabetic foot complications (FIG. 3, Table-4).

FIGS. 1,2 and 3 shows a significant increase in the Procalcitonin values with an increasing degree of severity of the DM II. The two groups of DM II patients, in particular, differ from the healthy controls. Surprisingly, the highest procalcitonin values were found in the DM II patients who already demonstrated diabetic foot complication.

Table-1, 2 and 4 shows not only Procalcitonin but also the parameters relevant to diabetes, such as glucose, HbAlc and leucocyte in the groups, in each instance.

Procalcitonin (PCT) levels were elevated in type 2 diabetic patients when compared with normal non-diabetic subjects. There was a statistically significant difference in serum procalcitonin of type 2 diabetic patients versus normal subjects (P≦0.01) (see Table. 3).

Procalcitonin levels were elevated in diabetic foot complication of patients when compared with normal non-diabetic subjects. There was a statistically significant difference in serum procalcitonin of diabetic foot complication of patients versus normal subjects (P≦0.001) (see Table. 5).

Tables

Table 1 shows not only procalcitonin but also the parameters relevant to diabetes, such as blood sugar (glucose), HbAlc and leucocyte count, in the diabetes patient groups, in each instance.

Table 2 shows not only procalcitonin but also glucose, HbAlc and leucocyte values, in the control (non-diabetic) groups, in each instance.

Table 3 shows PCT statistical differences of between diabetes patient and control (non-diabetic) groups.

Table 4 shows procalcitonin, glucose, HbA_{lc} and leucocyte values, in the diabetic foot patient groups, in each instance.

Table 5 shows PCT statistical differences of between diabetic foot patient and control (non-diabetic) groups.

TABLE 1
Diabetes patient group parameters: blood sugar,
procalcitonin, HbA_{1c} and leucocyte values.

| | Blood Sugar | Procalcitonin | | Leucocyte |
|---|---|---|---|---|
| Patient No: | (mg/dl) | (ng/ml) | HbA_{1c (%)} | (/mm³) |
| D-1 | 179 | 0.0489 | 8.1 | 6440 |
| D-2 | 248 | 0.0190 | 10.1 | 8480 |
| D-3 | 223 | 0.0291 | 9.4 | 5370 |
| D-4 | 123 | 0.0392 | 8.5 | 7890 |
| D-5 | 157 | 0.0190 | 7.0 | 7920 |
| D-6 | 158 | 0.0367 | 10.4 | 6330 |
| D-7 | 172 | 0.0553 | 7.5 | 9140 |
| D-8 | 304 | 0.0333 | 11.2 | 7770 |
| D-9 | 138 | 0.0190 | 7.2 | 5130 |
| D-10 | 370 | 0.0190 | 10.4 | 6440 |
| D-11 | 168 | 0.0710 | 8.0 | 8070 |
| D-12 | 155 | 0.0252 | 9.7 | 8880 |
| D-13 | 355 | 0.0409 | 11.6 | 5770 |
| D-14 | 170 | 0.0190 | 9.2 | 7700 |
| D-15 | 95 | 0.0464 | 7.2 | 8400 |
| D-16 | 107 | 0.0768 | 8.7 | 9400 |
| D-17 | 289 | 0.0650 | 9.3 | 8560 |
| D-18 | 144 | 0.0740 | 5.9 | 6800 |
| Arithmetic | 197.5 | 0.0409 | 8.85 | 7471.6 |
| average: | | | | |
| SD: | 82.5 | 0.02 | 1.56 | 1308.40 |

TABLE 2
Control group parameters: blood sugar, procalcitonin,
HbA_{1c} and leucocyte values.

| | Blood Sugar | Procalcitonin | | Leucocyte |
|---|---|---|---|---|
| Control No | (mg/dl) | (ng/ml) | HbA_{1c (%)} | (/mm³) |
| C-1 | 101 | 0.0224 | 6.1 | 5120 |
| C-2 | 102 | 0.0190 | 5.5 | 5880 |
| C-3 | 102 | 0.0269 | 6.2 | 6330 |
| C-4 | 89 | 0.0220 | 5.6 | 4740 |
| C-5 | 92 | 0.0190 | 5.8 | 5420 |
| C-6 | 96 | 0.0281 | 5.4 | 6270 |
| C-7 | 93 | 0.02 | 5.8 | 5970 |
| C-8 | 90 | 0.024 | 5.6 | 7890 |
| C-9 | 94 | 0.023 | 5.8 | 5220 |
| C-10 | 113 | 0.017 | 6.2 | 9200 |
| C-11 | 93 | 0.022 | 5.7 | 7300 |
| C-12 | 90 | 0.0190 | 5.7 | 7270 |
| C-13 | 91 | 0.0190 | 5.6 | 6300 |
| C-14 | 97 | 0.0260 | 5.6 | 6390 |
| C-15 | 80 | 0.028 | 6.1 | 5730 |
| Arithmetic | 94.86667 | 0.02236 | 5.78 | 6335.333 |
| Average: | | | | |
| SD: | 7.595738 | 0.0036 | 0.256905 | 1172.518 |

TABLE 3
PCT statistical differences of patient and control groups:

| | Control Group: | Patient Group: |
|---|---|---|
| | (Avg ± S.D) | (Avg ± S.D) |
| PCT(ng/ml) | 0.022 ± 0.0036 | 0.040933 ± 0.02** |

**p < 0.01

TABLE 4
Diabetic foot patient group parameters: blood sugar,
procalcitonin, HbA_{1c} and leucocyte values

| | Blood Sugar | Procalcitonin | | Leucocyte |
|---|---|---|---|---|
| Patient No | (mg/dl) | (ng/ml) | HbA_{1c (%)} | (/mm³) |
| DF-1 | 161 | 0.0704 | 13.6 | 9250 |
| DF-2 | 152 | 0.1378 | 6.3 | 6500 |
| DF-3 | 227 | 0.0472 | 8.5 | 7300 |
| DF-4 | 216 | 0.0582 | 9.1 | 6540 |
| DF-5 | 422 | 0.0227 | 14.2 | 9440 |
| DF-6 | 100 | 0.059 | 6.5 | 7410 |
| DF-7 | 100 | 0.0231 | 9.4 | 5610 |
| DF-8 | 135 | 0.0501 | 7.2 | 7640 |
| DF-9 | 169 | 0.0190 | 7.9 | 8840 |
| DF-10 | 148 | 0.0190 | 7.4 | 7570 |
| DF-11 | 74 | 0.2177 | 6.7 | 8590 |
| DF-12 | 111 | 0.0544 | 9.6 | 8210 |
| DF-13 | 69 | 0.0991 | 6.6 | 8720 |
| DF-14 | 128 | 0.0887 | 8.2 | 8060 |
| DF-15 | 190 | 0.028 | 11.8 | 9740 |
| Arithmetic | 160.1333 | 0.066293 | 8.866667 | 7961.333 |
| average: | | | | |
| SD: | 86.34885 | 0.053602 | 2.518408 | 1180.417 |

TABLE 5
PCT statistical differences of diabetic
foot patient and control groups:

| | Control Group: | Patient Group: |
|---|---|---|
| | (Avg ± S.D) | (Avg ± S.D) |
| PCT(ng/ml) | 0.022 ± 0.0036 | 0.066293 ± 0.05** |

**p < 0.01

### FIGURES

FIG. 1 shows the results of a graphical representation of diabetes patient group procalcitonin values;

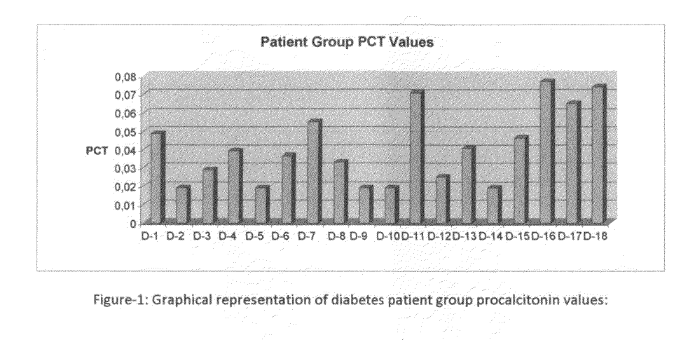

FIG. 2 shows the results of a graphical representation of control group procalcitonin values;

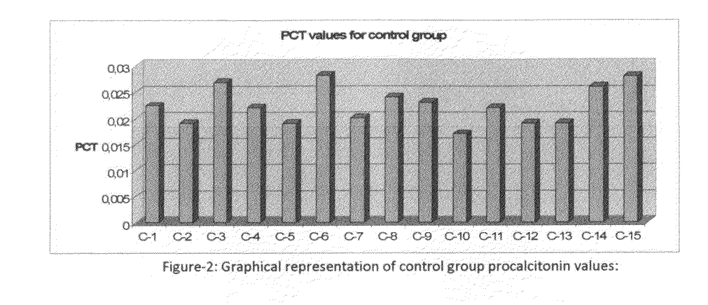

FIG. 3 shows the results of a graphical representation of diabetic foot patient group procalcitonin values;

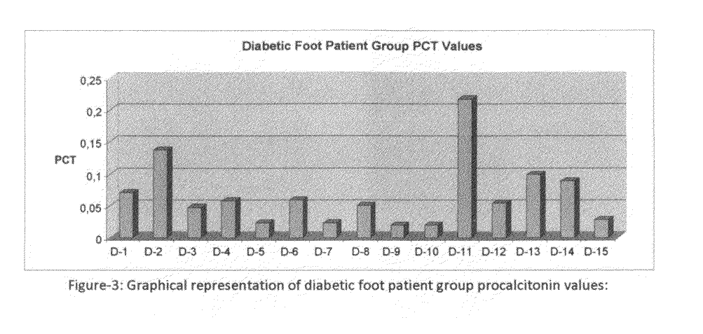

Sequence listing shows the results of procalcitonin (abbreviated as: “PCT”) with the related partial sequences.

1. Method for in vitro diagnosis and/or diabetic complications and/or risk stratification of diabetes mellitus, comprising determining procalcitonin (PCT: SEQ ID No. 1) or partial peptides or fragments thereof in a patient to be investigated.

2. Method according to claim 1, characterized in that in vitro diagnosis and/or risk stratification of Type II diabetes mellitus and its sequelae and concomitant illnesses, particularly endothelial dysfunction, hyperlipoproteinemia, hypertensive dysregulation of the cardiovascular system, diabetic retinopathy, nephropathy, renal insufficiency, neuropathy, diabetic foot syndrome and cardiovascular complications take place.

3. Method according to claim 1, further comprising determining at least one further marker selected from the group of inflammatory markers, vascular markers, and/or diabetic markers/factors in a patient to be investigated.

4. Method according to claim 3, characterized in that the inflammatory marker is selected from at least one marker of the group of C-reactive protein (CRP), cytokines, such as TNF-alpha, for example, interleukins, such as IL-6, interleukin-1.beta, Proadrenomedullin (proADM), midregional proadrenomedullin (MR-proADM), angiotensin II, endothelin-1.

5. Method according to claim 3, characterized in that the vascular marker is selected from at least one marker of the group of creatine kinase, myeloperoxidase, myoglobin, natriuretic protein, particularly ANP (or ANF), proANP, NT-proANP, BNP, proBNP, NT-proBNP, or (pro)hormones that regulate the cardiovascular system, such as pro-gastrin-releasing peptide (proGRP), pro-endothelin-1, pro-leptin, pro-neuropeptide-Y, pro-somatostatin, pro-neuropeptide-YY, pro-opiomelanocortin, or a partial sequence thereof, in each instance.

6. Method according to claim 3, characterized in that parallel or simultaneous determinations of the markers are carried out.

7. Method according to claim 1, characterized in that the determinations are carried out on at least one patient sample.

8. Method according to claim 1, characterized in that the determinations are carried out using an automated analysis device.

9. Method according to claim 1, characterized in that the determinations are carried out by means of a rapid test.

10. Method according to claim 1, characterized in that the diagnosis is for the stratification of patients for clinical decisions related to treatment or therapy of diabetes mellitus.

11. Method according to claim 1, characterized in that the diagnosis and/or risk stratification takes place for prognosis, for prophylaxis, for early detection and detection by means of differential diagnosis, for assessment of the degree of severity, and for assessing the course of diabetes mellitus, particularly Type II diabetes mellitus, and its concomitant illnesses and sequelae, as an accompaniment to therapy.

12. Diagnostic device for carrying out a method according to claim 1.

13. Kit for in vitro diagnosis and/or risk stratification of diabetes mellitus, particularly Type II diabetes mellitus, and its concomitant illnesses and sequelae, containing detection reagents for determining the marker procalcitonin (PCT: SEQ ID No. 1) or partial peptides or fragments thereof, or contained in a marker combination, wherein the marker combination contains other markers according to claim 3, and ancillary substances.


SEQUENCE LISTING (1 sequence)

SEQ ID NO: 1 - protein, 116 aa, Homo sapiens
APFRSALESSPADPATLSEDEARLLLAALVQDYVQMKASELEQEQEREGSSLDSPRSKRCGNLSTCMLGTYTQDFNKFHTFPQTAIGVGAPGKKRDMSSDLERDHRPHVSMPQNAN